(19) United States
(12) Patent Application Publication
(10) Pub. No.: US 2017/0119927 A1
(43) Pub. Date: May 4, 2017

(54) COMPOSITIONS AND METHODS FOR REPAIRING BONE

(71) Applicant: Biomet Manufacturing, LLC, Warsaw, IN (US)

(72) Inventors: Sonya Cooper, Miami, FL (US); Loan Song, Rancho Santa Margarita, CA (US)

(21) Appl. No.: 15/407,533

(22) Filed: Jan. 17, 2017

Related U.S. Application Data
(62) Division of application No. 14/163,449, filed on Jan. 24, 2014.
(60) Provisional application No. 61/756,846, filed on Jan. 25, 2013.

(51) Int. Cl. A61L 27/36

(52) U.S. Cl. CPC A61L 27/3608; A61L 27/365; A61L 2430/02

(57) ABSTRACT

Bone repair compositions comprising bone particles and periosteum tissue. The bone particles may be demineralized, and may comprise powder or fibers from cortical bone or from cancellous bone. The periosteal tissue can be micronized periosteal tissue, and may comprise periosteal powder, particulates, pieces, or combinations thereof. The bone repair composition can further comprise bone chips, mineralized cancellous bone, or additional materials. The present technology also provides methods of repairing a bone defect, comprising administering a bone repair composition to the site of the bone defect.

### INTRODUCTION

The present technology relates to a bone repairing composition and associated methods of production and use.

A bone repairing composition or filler may be used to correct defects caused by trauma, pathological disease, surgical intervention or other situations where defects need to be managed in osseous surgery. Bone reconstruction may be performed with various pastes, gels or putty-like materials containing a natural collagen or human cadaveric donor bone base. Such compositions may be prepared, for example, from demineralized allograft bone matrix (DBM).

DBM is typically provided for clinical use from “bone banks,” which harvest bone from human cadavers (donated and managed according to proper ethical and legal standards). Typically, DBM is prepared from cortical bone. The bone undergoes physical processing (such as grinding or shaping), and is then demineralized to form DBM. Because the bone may be harvested and processed in advance of its use, it is frequently dried (e.g., by lyophilization) and packaged under sterile conditions, for storage and shipping to the clinical site.

The sterile DBM is available in cubes, shavings or powder and is freeze-dried. Because the DBM is dry and difficult to manipulate, it may be made flowable or malleable with the addition of a wetting agent. For example, the blood of the subject to be treated may be mixed with the bone, bone powder or collagen. Use of blood or blood products may offer the benefits of being available at the operative site, being non-immunogenic to the patient, and providing proteins, monosaccharides, polysaccharides and glucoronic acid which increase the healing process and regeneration of bone. Other wetting agents include monosaccharides, disaccharides, water dispersible oligosaccharides, polysaccharides, low weight organic solvents, including glycerol, polyhydroxy compounds, such as mucopolysaccharide or polyuronic acid and various aqueous solutions. Regardless of the exact components, a primary goal in bone reconstruction is that the filler be highly effective in inducing bone formation, and that it become integrated at the application site.

### SUMMARY

The present technology provides bone repair compositions for application to a bone surface of a human or animal subject, comprising:
(a) bone particles; and
(b) periosteal tissue.
   In various embodiments, the bone particles are demineralized, and may comprise powder or fibers from cortical bone or from cancellous bone. The periosteal tissue can be micronized periosteal tissue, and may comprise periosteal powder, particulates, pieces, or combinations thereof. The bone repair composition can further comprise bone chips, mineralized cancellous bone, or additional materials. In some embodiments, the bone repair composition is lyophilized, and is hydrated prior to use in a physiologically acceptable liquid.

In some embodiments, the present technology provides a bone repair composition comprising:
(a) demineralized cortical bone powder, fibers, or combinations thereof; and
(b) periosteum powder, pieces, or combinations thereof.
   The bone repair composition may further comprise mineralized cancellous bone particles. Preferably, the bone repair composition comprises demineralized cortical bone powder, demineralized cortical bone fibers, mineralized cancellous bone particles, and periosteum pieces.

A method of manufacturing a bone repair composition is also provided. The method comprises:
(a) harvesting periosteal tissue from a bone;
(b) harvesting cortical bone from a bone; and
(c) combining the cortical bone and the periosteal tissue to generate a mixture of cortical bone and periosteal tissue.
   In various embodiments, harvesting cortical bone comprises processing cortical bone by milling, planing, or grinding cortical bone to generate cortical bone powder or fibers; and demineralizing the cortical bone powder or fibers to generate demineralized cortical bone powder or fibers. In some embodiments, the method comprises processing the periosteal tissue by immersing the periosteal tissue in 5 M sodium chloride solution for at least about 15 minutes, rinsing the periosteal tissue with water, removing excess water from the periosteal tissue, cutting the periosteal tissue into pieces, then combining the pieces of periosteal tissue with the demineralized cortical bone powder or fibers. In various embodiments, (a), (b), and (c) are performed simultaneously by obtaining cortical bone comprising periosteal tissue; and grinding the cortical bone comprising periosteal tissue into a powder or milling the cortical bone comprising periosteum tissue into fibers to generate a mixture of cortical bone particles, cortical bone-periosteal tissue particles, and periosteum pieces. The bone repair composition can be stored at low temperatures (e.g., −80° C.), with or without a cryopreservation solution, it can be lyophilized and stored, or it can be presented to an end user “as is.”

The present technology also provides methods of repairing a bone defect, comprising:
(a) obtaining a bone repair composition comprising bone particles and periosteal tissue; and
(b) administering the bone repair composition to the site of the bone defect.
   In various embodiments, obtaining the bone repair composition comprises mixing bone particles and periosteal tissue. The bone particles comprise demineralized cortical bone powder, fibers, or combinations thereof, and the periosteal tissue comprises micronized periosteum powder, periosteum pieces, or combinations thereof. The bone repair composition can be hydrated with a physiologically acceptable liquid, or it can be administered in its non-hydrated form.

### DRAWINGS

FIGS. 1(a)-1(c) are photographs of Composition A, an exemplary bone repair composition of the present technology;

FIGS. 2(a)-2(c) are photographs of Composition B, an exemplary bone repair composition of the present technology;

FIGS. 3(a)-3(c) are photographs of Composition C, an exemplary bone repair composition of the present technology; and

FIGS. 4(a)-4(c) are photographs of Composition D, an exemplary bone repair composition of the present technology.

It should be noted that the figures set forth herein are intended to exemplify the general characteristics of materials, compositions, and methods among those of the present technology, for the purpose of the description of certain embodiments. These figures may not precisely reflect the characteristics of any given embodiment, and are not necessarily intended to fully define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom.

The present technology provides a bone repair composition compositions for application to a bone surface of a human or animal subject, comprising:
(a) bone powder; and
(b) periosteal tissue powder.
   The compositions of this technology may be provided in any form suitable for implantation onto bone. In various embodiments, the compositions are in the form of a maleable putty or paste.

Bone used in the compositions may be obtained from any physiologically appropriate source. In some embodiments, bone is obtained from non-human animal sources (i.e., for xenogenic implantation in a human subject) such as cows and pigs. Preferably, however, for human use, bone is obtained from human cadavers (i.e., for allogeneic implantation in a human subject), following appropriate ethical and legal requirements. Such human bone material is available from a variety of tissue banks. Appropriate processing controls useful in manufacturing the compositions of this technology are well known and practiced in the tissue banking industry. Such controls include those practiced in conjunction with the processing described further herein, as appreciated by one of ordinary skill in the art.

The bone may comprise cortical bone, cancellous bone, or a combination thereof. Cancellous bone is available in a range of porosities based on the location in the body from which the bone is harvested. Highly porous cancellous bone may be harvested from various areas such as the iliac crest, while less porous bone may be harvested from areas such as the tibial condyle femoral head, and calcaneus. Cortical bone may be obtained from long bones, such as the diaphyseal shaft of the femur and tibia. A preferred implant comprises cortical bone.

### Bone Powder

Bone powder useful herein preferably has a size of 5000 microns or less. In various embodiments, the bone powder ranges from about 50 microns to about 1500 microns, from about 100 microns to about 1000 microns, from about 125 to about 800 microns, or from about 150 to about 600 microns.

In various embodiments, the bone powder is demineralized. As referred to herein, “demineralized bone” refers to any bone material from which a substantial portion of naturally-occurring minerals has been removed, in whole or in part. The mineral content of the demineralized bone may be less than about 20%, optionally less than about 10%, optionally less than about 5%. In some embodiments, the mineral content is from 0% to about 5% or from about 0.5% to about 3% or from about 0.5% to about 2%. (As referred to herein, all percentages are by weight unless otherwise specified.)

The bone may be demineralized using any of a variety of methods, including those known in the art using acids, cheating agents, or electrolysis. Preferred chemical treatments include those using hydrochloric acid, ethylene diamine tetraacetic acid (EDTA), peracetic acid, or citric acid. Demineralization techniques among those useful herein are described in K. U. Lewandrowski et al., “Kinetics of cortical bone demineralization:controlled demineralization—a new method for modifying cortical bone allografts,” J Biomed. Mater. Res., 31: 365-372 (1996); K. U. Lewandrowski, et al., “An electron microscopic study on the process of acid demineralization of cortical bone,” Cal. Tiss. Int., 61: 294-297 (1997); and K. U. Lewandrowski, et al., “Improved osteoinduction of cortical bone allografts: a study of the effects of laser perforation and partial demineralization,” J Orthop. Res., 15: 748-756 (1997); and Reddi et al., “Biochemical sequences in the transformation of normal fibroblasts in adolescent rats”, Proc. Nat. Acad. Sci., 69 pp. 1601-5 (1972).

The demineralization treatment provides a DBM comprising insoluble collagen and other non-collagenous proteins such as bone growth factors including bone morphogenetic proteins (BMPs). Such compositions comprise DBM that has not undergone treatments that destroy the chemical composition of collagen, or denature bone growth factors within the DBM. Such DBM to be irradiated does not comprise added osteogenic proteins. As referred to herein, “osteogenic proteins” are proteins that are capable of producing a developmental cascade of cellular events resulting in endochondral bone function. Such osteogenic proteins are those referred to in the art as osteogenic proteins, osteoinductive proteins and bone morphogenetic proteins. The DBM may, however, include non-osteogenic proteins and active materials, such as those selected from the group consisting of synthetic and recombinant growth factors, growth factor mimetics, morphogens, and plasmid and viral vectors. Products made using such compositions may further comprise added osteogenic proteins and such non-osteogenic proteins and active materials added to the irradiated DBM after irradiation. In some embodiments, methods of this technology comprise the irradiation of bone material consisting essentially of DBM. Such methods are described in U.S. Pat. No. 7,678,385, Reddi, issued Mar. 16, 2012.

The bone may be subjected to defatting/disinfecting and acid demineralization treatments. A preferred defatting/disinfectant solution is an aqueous solution of a lower alcohol, such as ethanol. Preferably, the defatting/disinfection solution contains at least about 10% to 40% water (i.e., about 60% to 90% defatting agent such as alcohol). For example, the solution may contain from about 60% to about 85% alcohol. Following defatting, the bone can be immersed in acid or chelating agent over time to effect demineralization. The concentration of the acid or chelating agent in such demineralization operation is preferably about 0.5N to about 1.0N, with demineralizing time being from about 2 to about 12 hours under ambient conditions.

### Bone Fibers

Bone, especially cortical bone, can be processed into long, thin fibers, which are useful herein. Preferably, the fibers have a length of from about 1 mm to about mm. More preferably, the fibers have a length of from about 1 mm to about 10 mm. The bone fibers can be mineralized or demineralized. Preferably, the bone fibers are demineralized cortical bone fibers (DBM fibers).

Bone fibers are typically processed in parallel to natural fibers of long bones and can be prepared by a variety of methods. A first method of preparing bone fibers comprises obtaining a mineralized or demineralized fibrous bone composition; and pulverizing or crushing the fibrous bone composition until fibers of from about 1 mm to about 15 mm are generated. Preferably, the fibrous bone composition is demineralized. Bone fibers may be obtained from commercially-available demineralized fibrous bone composition, such as FiberStack™ DBM (Biomet, Warsaw, Ind.). However, preferably, the bone fibers are obtained, along with periosteal tissue, from a single bone sample. Where a mineralized fibrous bone composition is utilized for preparing bone fibers, the resulting bone fibers can be demineralized by any method commonly used in the art, including those described above.

Another method of preparing bone fibers comprises obtaining a bone fragment and cutting the bone fragment with a side of a mill cutter to generate long slender fibers. In this method, an axis of the mill cutter is parallel to an axis of the bone fragment being cut. Preferably, the bone fragment is from a long bone. More preferably, the bone fragment is cortical bone from a long bone. The bone fibers can be mineralized or demineralized. Where demineralized bone fibers are desired, demineralization can be performed by any method commonly used in the art, including those described above.

### Cancellous Bone

In various embodiments, particles of cancellous bone are useful herein. In some embodiments, the cancellous bone is mineralized, and in other embodiments, the cancellous bone is demineralized. Demineralized or mineralized cancellous bone particles can be obtained from a manufacturer or they can be prepared by various methods, such as, for example, by crushing or pulverizing cancellous bone. Where demineralized bone fibers are desired, demineralization can be performed by any method commonly used in the art, including those described above. Alternatively, cancellous bone can be prepared by obtaining mineralized or demineralized cancellous bone from a manufacturer, and crushing or pulverizing the cancellous bone. Indux™ Cancellous Sponge and Indux™ Cancellous Strip (Biomet, Warsaw, Ind.), are non-limiting examples of demineralized cancellous bone available from a manufacturer. However, preferably, the cancellous bone is obtained, along with periosteal tissue, from a single bone sample. Preferably, the particles of cancellous bone have a diameter of from about 250 μm to about 7 mm. More preferably, the particles of cancellous bone have a diameter of from about 1 mm to about 4 mm.

### Periosteal Tissue

Periosteal tissue useful herein includes the membrane of tissue that lines the outer surface of bones. In general, such tissue includes the outer “fibrous lay”, the “cambium layer” for “osteogenic layer”), and combinations thereof. Periosteal tissue may be removed from bone by any appropriate means, including scraping with a sharp tool or blade or by abrading with a wire brush. It can be mechanically minced by chopping or slicing with shears or knife blades to make small particles.

The periosteal tissue may be in any physical form suitable for the desired rheological and handling characteristics for implantation at a site of bone repair. For example, the periosteal tissue may comprise periosteal powder, particulate, pieces, or combinations thereof.

In various embodiments, the periosteal tissue comprises a powder or particulate. In such embodiments, the periosteal tissue may be ground to obtain a particle size of 1200 microns or less. Suitable techniques include those known in the art, such as cryomilling. In various embodiments, the periosteal tissue has a mean particle size less than about 1000 microns, less than about 800 microns, or less than about 500 microns, or less than about 400 microns.

In various embodiments, the periosteal tissue is in the form of pieces. Periosteum pieces can be generated by harvesting a portion of periosteal tissue from a bone and cutting the periosteal tissue into pieces with a scissors. The shapes of the periosteum pieces are not restricted. For example, the periosteum pieces can be in the form of strings, blocks, sheets, patches, irregular shapes, or combinations thereof. For example, in some embodiments the periosteum pieces are sheets or patches measuring from about 0.5 mm to about 8 mm, or from about 1 mm to about 5 mm in length and in width. In various embodiments, the periosteum pieces have a volume of from about 1 mm³ to about 50 mm³, or from about 2 mm³ to about 20 mm³ Preferably, the periosteum pieces have a volume of from about 5 mm³ to about 15 mm³.

When mixed with bone graft materials, such as DBM powder or fibers, the periosteum pieces stick to the bone graft materials and to each other. Thus, a continuous network of overlapping clusters of periosteum-graft materials is created. Overlapping clusters can prevent the resulting composition from separating during bending and stretching.

### Additional Materials

The compositions of the present technology may comprise additional, optional materials. For example, the compositions can include bone graft substitutes such as bioactive glass, glycerol, or combinations thereof.

In some embodiments the compositions of the present technology include one or more bone graft substitute, such as calcium carbonate or calcium phosphate. An example of a bone graft substitute is granules of a resorbable osteoconductive matrix comprising hydroxyapatite and calcium carbonate. Preferably, the granules have a diameter of from about 0.5 mm to about 1 mm. ProOsteon® 200R and ProOsteon® 500R (Biomet, Warsaw, Ind.) are non-limiting examples of resorbable osteoconductive matrices comprising hydroxyapatide and calcium carbonate that are available as granules.

The compositions of the present technology may also comprise particles or beads of bioactive glasses. Bioactive glasses are glass or ceramic biomaterials that are biocompatible with human and non-human bodies. As appreciated by a person of ordinary skill in the art, a bioactive glass is known as “Bioglass.” Bioglass is composed of SiO₂, P₂O₅, CaO, Ca(PO₃)₂, CaF₂, and Na₂O in various combinations and proportions. Some Bioglasses further comprise B₂O₃, Fe₂O₃, MgO, K₂O, Al₂O₃, and Ta₂O₅/TiO₂ in various combinations and proportions. Many Bioglasses are known in the art; non-limiting examples of which include Bioglass 42S5.6, Bioglass 46S5.2, Bioglass 49S4.9, Bioglass 52S4.6, Bioglass 55S4.3, Bioglass 60S3.8, Bioglass 45S5, Bioglass 45S5F, Bioglass 45S5.4F, Bioglass 40S5B5, Bioglass 52S4.6, Bioglass 55S4.3, and Bioglass 8625.

In various embodiments, the composition may additionally comprise non-demineralized bone powder or chips. The non-demineralized bone powder or chips can be cortical bone, cancellous bone, or combinations thereof. Such bone chips may have a size of from about 750 to about 2000 microns, preferably from about 750 to about 1500 microns. In other embodiments, the composition further comprises additional materials, such as calcium carbonate, calcium phosphate, glycerol, Bioglass, a bone graft substitute, or combinations thereof.

In some embodiments, the compositions optionally comprise a biocompatible carrier or physiologically acceptable liquid. A physiologically acceptable liquid is a liquid that is biocompatible with human and non-human bodies. Such carriers and liquids include saline, phosphate buffered saline (PBS), hyaluronic acid, cellulose ethers (such as carboxymethyl cellulose), collagen, gelatin, autoclaved bone powder, osteoconductive carriers, whole blood, blood fractions, bone marrow aspirate, concentrated bone marrow aspirate, and mixtures thereof. Non-limiting examples of blood fractions include serum, plasma, platelet-rich plasma, concentrated platelet-rich plasma, platelet-poor plasma, and concentrated platelet poor plasma. In other compositions, however, the compositions are essentially free of such carriers, and are preferably free of materials not obtained from allogeneic tissue. Thus, the present technology provides, in some embodiments, compositions consisting essentially of DBM and periosteal tissue.

### Compositions and Manufacturing

The compositions of the present technology comprise DBM and periosteal tissue at various levels, for example including either material at a level of from 1% to about 99%, by weight. In some embodiments, either material is present at a level of from about 25% to about 75%, from about 30% to about 65%. Accordingly, the present technology provides broad mixing ratios of DBM particles and periosteal tissue. For example, the DBM particles:periosteal tissue ratio can be from about 100:1, to about 1:100. In a preferred embodiment, the DBM particles:periosteal tissue ratio is about 1.5:1. For example, the composition may comprise about 1.5 g (60%) DBM particles and about 1.0 g (40%) periosteal powder. The DBM particles can be bone powder, bone fibers, or combinations thereof.

The compositions of the present technology may be made using any appropriate technique for mixing the demineralized bone material and periosteal tissue. For example, in some embodiments, DBM powder and periosteum powder are mixed while both are in a dry form. As further described below, the composition may be provided directly for use in a surgical procedure, without further processing, wherein the mixture of powders is rehydrated to form a paste or putty during the surgical procedure. Therefore, in some embodiments, a mixture of DBM powder and periosteal tissue is placed at a surgical site and allowed to hydrate with autologous fluids in situ.

Accordingly, the present technology provides a method for manufacturing a bone repair composition. The method comprises (a) harvesting periosteal tissue from a bone; (b) obtaining cortical bone; and (c) combining the cortical bone and the periosteal tissue to generate a mixture of cortical bone and periosteal tissue, wherein the mixture is the bone repair composition. In various embodiments, obtaining cortical bone comprises processing cortical bone by milling, planing, or grinding to generate cortical bone powder or fibers. The cortical bone powder or fibers can then be demineralized by any means commonly used in the art to generate DBM powder or DBM fibers.

In some embodiments, the harvested periosteal tissue is processed. Processing the periosteal tissue includes immersing the periosteal tissue in a 5 M sodium chloride solution for at least about 15 minutes, removing the periosteal tissue from the sodium chloride solution, rinsing the periosteal tissue with water a plurality of times (preferably at least 3 times), removing excess water from the periosteal tissue (e.g., with absorbent lint-free wipes), and cutting the periosteal tissue into pieces. Cutting can be performed with a razor blade, scissors, or any other suitable instrument.

In some embodiments, the method further comprises determining a DBM powder:DBM fiber ratio. The DBM powder:DBM fiber ratio can be from about 1:100 to about 100:1. Additionally, the method may comprise a step of determining a DBM powder/fiber:periosteal tissue ratio. As described above, the DBM powder/fiber:periosteal tissue ratio can be from about 100:1 to about 1:100.

In some embodiments, the present technology provides a method for manufacturing a bone repair composition comprising: (a) obtaining a bone fragment comprising cortical bone and periosteal tissue; and (b) milling, planing, or grinding the bone fragment into a suitable form, e.g., powder or fibers. This method allows the cortical bone and periosteum to be processed simultaneously, which saves time and is cost efficient. The bone repair composition manufactured by this method comprises a combination of cortical bone particles, bone-periosteum particles, and periosteum pieces. The combination of cortical bone particles, bone-periosteum particles, and periosteum pieces can then be processed. Processing the combination includes immersing the combination in a 5 M sodium chloride solution for at least about 15 minutes, removing the periosteal tissue from the sodium chloride solution, rinsing the periosteal tissue with water a plurality of times (preferably at least 3 times), and optionally removing excess water from the periosteal tissue with absorbent lint-free wipes. The combination of cortical bone particles, bone-periosteum particles, and periosteum pieces can then be demineralized with from about 0.5 N to about 0.6 N hydrochloric acid (HCl).

Regardless of the method followed, the bone repair composition manufactured by the present methods can be stored at −80° C. in a cryopreservation solution, it can be lyophilized and stored, or it can be presented to an end user “as is.” The end user is typically a medical doctor, nurse, or other trained medical technician.

In some embodiments, the composition may be hydrated with saline, PBS or other physiologically acceptable fluid, and provided for use in a hydrated form. Further, the hydrated composition may be dried, such as by lyophilization. The compositions may also be sterilized, using techniques among those known in the art. A mixture of DBM powder/fibers and periosteal tissue, including a lyophilized mixture, can placed at a surgical site directly, or it can be rehydrated with saline, PBS or other physiologically acceptable fluid, to form a wet paste.

In some embodiments, the compositions of the present technology are formed into a product/article or into a particular shape, either with the use of a carrier, or a binder. For example, the article or shape could be a sheet, a disc or other flat plate, an elongated member, such as a bar or rod, a bone-shaped member suitable for the intended bone repair procedure. The composition may be shaped during a bone repair procedure into a plug, ball or other form suitable for implantation into a pocket, recess, bore or other receiving surface of the implantation site.

### Methods of Use

The present technology also provides methods for repairing a bone defect using a composition as described herein, i.e., a method of repairing a bone defect comprising administering to the site of the defect a composition comprising bone particles and periosteal tissue. As used herein, a “bone defect” refers to bone imperfections caused by birth defect, injury, trauma, disease, decay, or surgical intervention, and the desired repair may be for cosmetic or therapeutic reasons. A “surgical site” is a site on a subject at which surgery is performed, including a bone defect that requires repair.

For example, bone repairing compositions of the present technology may be used to correct bone defects in orthopedic, neurosurgical plastic or reconstructive surgery, in periodontal procedures, and in endodontic procedures. Examples include repair of simple and compound fractures and non-unions, external and internal fixations, joint reconstructions such as arthrodesis, general arthroplasty, cup arthroplasty of the hip, femoral and humeral head replacement, femoral head surface replacement and total joint replacement, repairs of the vertebral column including spinal fusion and internal fixation, tumor surgery, e.g. deficit filling, discectomy, laminectomy, excision of spinal cord tumors, anterial cervical and thoracic operations, repair of spinal injuries, scoliosis, lordosis and kyphosis treatments, intermaxillary fixation of fractures, mentoplasty, temporomandibular joint replacement, alveolar ridge augmentation and reconstruction, inlay bone grafts, implant placement and revision, sinus lifts, etc.

In some embodiments, a method of repairing bone comprises: (a) obtaining a bone repair composition comprising bone particles and periosteal tissue; and (b) administering the bone repair composition to a surgical site. In various embodiments, obtaining a bone repair composition comprises mixing bone particles and periosteal tissue in order to generate the bone repair composition, as described above.

In other embodiments, obtaining a bone repair composition comprises selecting a pre-manufactured bone repair composition. The pre-manufactured bone repair composition can be manufactured according to the methods described above. The pre-manufactured bone repair composition should comprise a desired bone particles:periosteal tissue ratio, to ensure that a desired consistency will be obtained. In various embodiments, the bone repair composition is stored in a cryopreservative composition. Prior to use, the cryopreservative composition should be removed, and the bone repair composition should be washed with water, saline, phosphate buffered saline, or other appropriate wash solution. Excess wash solution can be removed from the bone repair composition if desired.

A physiologically acceptable liquid, preferably containing water, may be added to the bone repair composition prior to placement into the site or defect. Such physiologically acceptable liquids include those discussed above, including physiological saline or a blood product. Blood products include whole blood and blood fractions such as platelet rich plasma and platelet poor plasma.

In some embodiments, the bone repair composition is hydrated with a physiologically acceptable liquid and biocompatible carrier. Non-limiting examples of physiologically acceptable liquids include saline, phosphate buffered saline (PBS), hyaluronic acid, cellulose ethers such as carboxymethyl cellulose), collagen, gelatin, autoclaved bone powder, osteoconductive carriers, whole blood, blood fractions, bone marrow aspirate, concentrated bone marrow aspirate, and mixtures thereof. Non-limiting examples of blood fractions include serum, plasma, platelet-rich plasma, concentrated platelet-rich plasma, platelet-poor plasma, and concentrated platelet poor plasma. After hydrating, the bone repair composition becomes a putty or a paste that can be molded into a predetermined shape or administered to a bone defect and manipulated to conform to the bone defect in such a manner that will promote healing. For example, the composition may be hydrated with about 2 ml of saline blood per 2.5 g of combined DBM and periosteal powder.

In some embodiments, the bone repair composition is not hydrated prior to administering. Therefore, it is administered to the surgical site, where it is allowed to hydrate with autologous fluids in situ.

Methods and compositions among those of the present technology are illustrated in the following non-limiting examples.

### EXAMPLES

Table 1 shows various compositions and formulations that were prepared according to various methods. Composition A was prepared by combining DBM powder, micronized periosteal tissue and mineralized cancellous bone. Compositions B, C, and D, were prepared by combining various combinations and amounts of DBM powder, DBM fibers, periosteum pieces, mineralized cancellous bone, and bone graft substitute. Composition E was prepared by grinding bone containing periosteum into a mixture of bone, bone-periosteum, and periosteum pieces. Composition F was prepared in the same manner as Composition E, with the exception that the mixture of bone, bone-periosteum, and periosteum pieces was combined with additional DBM powder. All the compositions have the consistency of a putty.

TABLE 1
Bone Repair Compositions

| | DBM | DBM | | Periosteum | Mineralized | |
|---|---|---|---|---|---|---|
| | Powder | Fibers | Micronized | Pieces (1 × 1 mm | Cancellous | Bone Graft |
| Composition | (125-500 mm) | (1-10 mm) | Periosteum | to 5 × 5 mm) | Bone (1-4 mm) | Substitute* |
| A | 0.5 g | — | 0.5 g | — | 0.3 g | — |
| B | 0.5 g | — | — | 0.5 g | 0.3 g | — |
| C | 0.5 g | — | — | 0.5 g | — | 0.3 g |
| D | 0.5 g | 0.1 g | — | 0.55 g | 0.3 g | — |
| E | — | Yes** | — | Yes** | 0.5 g | — |
| F | 0.5 g | Yes*** | — | Yes*** | 0.5 g | — |

*ProOsteon ® 200R granules (0.5-1 mm)
**1.5 g DBM fibers-Periosteum (0.5-1.5 cm)
***1.0 g DBM fibers-Periosteum (0.5-1.5 cm)

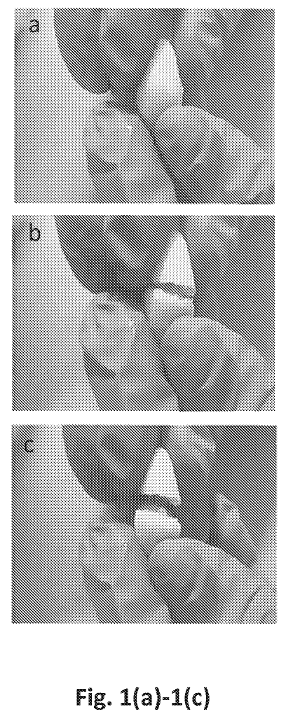

The characteristics of Composition A are demonstrated in FIG. 1(a)-1(c). FIG. 1(a) shows that Composition A was molded by hand. In FIG. 1(b), the putty was stretched lightly by hand, wherein the putty was completely separated into two parts. In FIG. 1(c) the putty was stretched further by hand to show the separation surface.

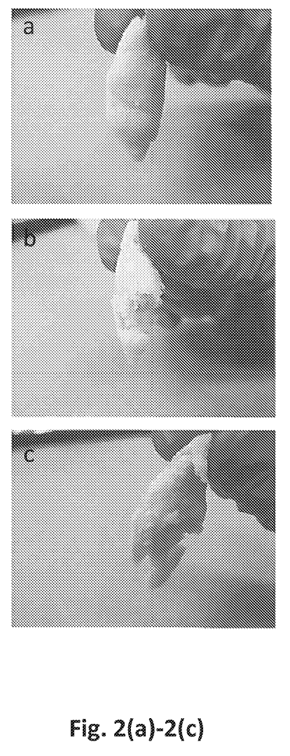

The characteristics of Composition B are demonstrated in FIG. 2(a)-2(c). FIG. 2(a) shows that Composition B was molded by hand and held upside down. In FIG. 2(b), the putty was stretched lightly by hand and held downward on one end. The putty was able to maintain its shape after being stretched. In FIG. 2(c), the putty was stretched further by hand and held down on one end. Still, the putty was able to maintain its shape.

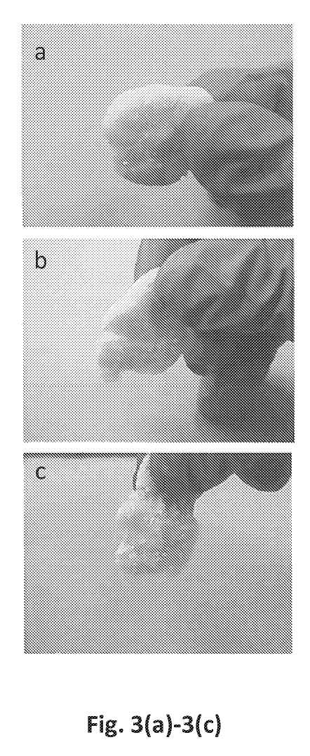

The characteristics of Composition C are demonstrated in FIG. 3(a)-3(c). FIG. 3(a) shows that the Composition C was molded by hand into a disc or pancake-like shape. In FIG. 3(b), the putty was stretched lightly by and held downward on one end. The putty was able to maintain its shape after being stretched. In FIG. 3(c), Composition C was stretched further by hand and held downward on one end. Still, the putty was able to maintain its shape.

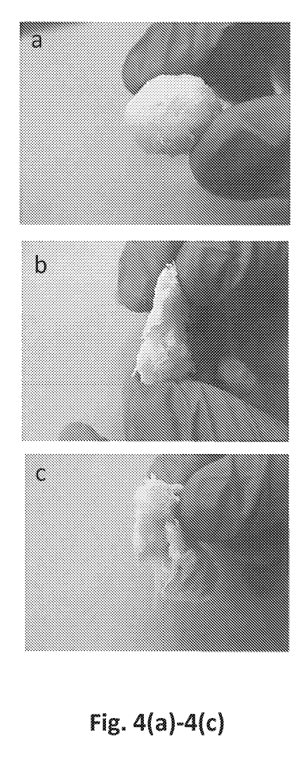

The characteristics of Composition D are demonstrated in FIG. 4(a)-4(c). FIG. 4(a) shows that Composition D was molded by hand into a ball shape. In FIG. 4(b), the putty was stretched lightly by hand. As shown in FIG. 4(b), the putty was able to maintain its shape after being stretched. In FIG. 4(c), the putty was stretched further by hand and held downward on one end. Again, the putty was able to maintain its shape after being stretched.

Photographs of Compositions E and F are not shown. Nonetheless, they both demonstrated good moldability, flexibility, and disintegration qualities.

### Non-limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The disclosure of all patents and patent applications cited in this disclosure are incorporated by reference herein.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

What is claimed is:

1. A bone repair composition comprising:
(a) bone particles; and
(b) periosteal tissue.
